(19) United States
(12) Patent Application Publication
(10) Pub. No.: US 2011/0135608 A1
(43) Pub. Date: Jun. 9, 2011

(54) USE OF PROBIOTIC BACTERIA FOR THE TREATMENT OF HYPERHOMOCYSTEINAEMIA

(75) Inventors: Giovanni Mogna, Novara (IT); Gian Paolo Strozzi, Novara (IT)

(73) Assignee: PROBIOTICAL S.P.A., I-28100 Novara (NO) (IT)

(21) Appl. No.: 12/993,037

(22) PCT Filed: Apr. 8, 2009
(86) PCT No.: PCT/EP09/54221

§ 371 Date: Dec. 9, 2010

(30) Foreign Application Priority Data
May 16, 2008 (IT) MI2008A000898

(51) Int. Cl. A61K 35/74; A61P 3/00

(52) U.S. Cl. 424/93.4

(57) ABSTRACT

The present invention refers to the use of probiotic bacteria for the treatment of hyperhomocysteinaemia. The present invention furthermore refers to a composition comprising probiotic bacteria in association with at least one vitamin chosen from the group comprising the B group of vitamins for the treatment of plasma hyperhomocysteinaemia.

The present invention refers to the use of probiotic bacteria for the treatment of hyperhomocysteinaemia. The present invention furthermore refers to a composition comprising probiotic bacteria in association with at least one vitamin chosen from the group comprising the B group of vitamins for the treatment of plasma hyperhomocysteinaemia.

In Italy and in all the developed countries, certain illnesses of the metabolism are rapidly on the increase, such as obesity, diabetes, arterial hypertension, cardiovascular diseases and systemic degenerative and neurodegenerative illnesses.

In addition to genetic predisposition, triggering factors are to be found in the alimentary regimes and lifestyles of “advanced” societies which entail a high consumption of saturated fats and refined foods, a low intake of soluble and insoluble fibres, sedentary habits, smoke, environmental pollution and, in general, stressful living conditions.

In a statistical sense, a person affected by a metabolic illness has not only a reduced life expectancy but also a worse quality of life.

Some of these illnesses, such as Alzheimer's and Parkinson's disease, are destined to increase further in parallel with the population's increase in the life expectancy. This phenomenon already constitutes a serious social and economic cost not only to the families of the sufferers, but also to national health services.

In the case of cardiovascular and neurodegenerative diseases, the scientific community has highlighted the existence of a warning parameter, homocysteine, of which a high concentration in the blood constitutes a proven risk factor for stroke, occlusive arterial pathology, venous thrombosis, atherosclerotic cardiovascular disease and probably for Alzheimer's disease and vascular dementias. High concentrations of homocysteine are in the majority of cases to be attributed to a nutritional deficiency resulting from an unbalanced and/or incomplete diet.

The necessity therefore remains of dealing with the pathologies resulting from high concentrations of plasma homocysteine in order to improve the quality of life of people affected by metabolic illnesses, such as hyperhomocysteinaemia.

In particular, there remains a need reduce the concentration of plasma homocysteine, which is responsible for serious illnesses and pathological conditions.

Finally, there remains the need to have available a formulation designed to reduce high concentrations of homocysteine in the plasma.

The Applicant has selected a number of strains of bacteria capable of providing a valid response to the needs present in the state of the art.

One object of the present invention relates to a bacterial strain chosen from the group consisting of Bifidobacterium adolescentis No. DSM 16594, Bifidobacterium adolescentis No. DSM 16595, Bifidobacterium breve No. DSM 16596, Bifidobacterium pseudocatenulatum No. DSM 16597, Bifidobacterium pseudocatenulatum DSM 16598, Bifidobacterium adolescentis No. DSM 18350, Bifidobacterium adolescentis No. DSM 18352, Bifidobacterium adolescentis No. DSM 18353 and Bifidobacterium pseudocatenulatum No. DSM 21444 for use in the treatment of plasma hyperhomocysteinaemia.

Another object of the present invention relates to the use of at least one of the above-mentioned strains for the preparation of a pharmaceutical composition for the treatment of plasma hyperhomocysteinaemia.

The Applicant has found that the combination of at least one particular selected strain of bacteria with at least one vitamin chosen from the group comprising the B group of vitamins is capable of reducing and normalising levels of plasma hyperhomocysteinaemia.

The association of a vitamin, chosen from the group comprising the B group of vitamins (vitamins immediately available to the organism) and folic acid produced in situ by the selected strains of bacteria, ensures greater efficiency in the reduction and normalisation of levels of plasma homocysteine.

Therefore, an object of the present invention relates to a composition comprising at least one of the above-mentioned bacterial strains in association with at least one vitamin chosen from the group comprising the B group of vitamins for the treatment of plasma hyperhomocysteinaemia.

Another object of the present invention relates to the use of at least one of the above-mentioned bacterial strains in association with at least one vitamin chosen from the group comprising the B group of vitamins for the preparation of a pharmaceutical composition for the treatment of plasma hyperhomocysteinaemia.

Other preferred embodiments of the present invention are cited below and claimed in the attached dependent claims.

In a preferred embodiment, the B group of vitamin is chosen from the group comprising vitamin 52 (riboflavin 5-phosphate sodium), vitamin B6 (pyridoxine hydrochloride), vitamin B9 (folic acid) and vitamin 512 (cyanocobalamine).

In the context of the present invention, folic acid and folates are often used as synonyms, identifying a series of compounds which have a common vitaminic activity and the same basic chemical formula which comprises one molecule of pteroic acid and one or more molecules of glutamic acid (pteroyl-monoglutamate folic acid or pteroyl-polyglutamate folic acid). The Applicant has isolated and characterised folic acid-producing strains belonging to GRAS (Generally Recognised as Safe) species. The Applicant has identified and characterised certain strains of Bifidobacterium capable of producing “in vitro” folic acid.

### “In Vitro” Study

Screening and quantitative determination of the folic acid produced by the strains under examination were performed by means of a microbiological assay, by turbidimetrically assessing the development of other bacterial species (such as Enterococcus hirae ATCC 8043) whose growth is a function of the quantity of folic acid present in the broth medium used.

From this first phase of the work, 9 strains of bacteria capable of producing folic acid were identified and deposited by the Applicant with the DSMZ International Collection in Germany.

| | Deposited | Number | | |
|---|---|---|---|---|
| | at | of | Date of | |
| Bacterium | Institute | deposit | deposit | Owner |
| Bifidobacterium | DSMZ | DSM | Jul. 21, 2004 | Probiotical |
| adolescentis BA 03 | | 16594 | | S.p.A. |
| Bifidobacterium | DSMZ | DSM | Jul. 21, 2004 | Probiotical |
| adolescentis BA 04 | | 16595 | | S.p.A. |
| Bifidobacterium | DSMZ | DSM | Jul. 21, 2004 | Probiotical |
| breve BR 04 | | 16596 | | S.p.A. |
| Bifidobacterium | DSMZ | DSM | Jul. 21, 2004 | Probiotical |
| pseudocatenulatum BP | | 16597 | | S.p.A. |
| 01 | | | | |
| Bifidobacterium | DSMZ | DSM | Jul. 21, 2004 | Probiotical |
| pseudocatenulatum BP | | 16598 | | S.p.A. |
| 02 | | | | |
| Bifidobacterium | DSMZ | DSM | Jun. 15, 2006 | Probiotical |
| adolescentis EI 3 | | 18350 | | S.p.A. |
| Bifidobacterium | DSMZ | DSM | Jun. 15, 2006 | Probiotical |
| adolescentis EI 18 | | 18352 | | S.p.A. |
| Bifidobacterium | DSMZ | DSM | Jun. 15, 2006 | Probiotical |
| catenulatum EI 20 | | 18353 | | S.p.A. |
| Bifidobacterium | DSMZ | DSM | May 13, 2008 | Probiotical |
| pseudocatenulatum B | | 21444 | | S.p.a. |
| 660 | | | | |

For the strain DSM 21444 the conditions of cultivation are as follows: the medium—TPY trypticase 10 g/l, phytone 5 g/l, yeast extract 5 g/l, glucose 10 g/l, tween 80 1 ml/l, K₂HPO₄ 2 g/l, MgCl₂ 0.5 g/l, ZnSO₄ 0.25 g/l, CaCl₂ 0.15 g/l, L-Cysteine hydrochloride 1-hydrate 0.5 g/l. Sterilisation is performed for 15 minutes at 121° C. at an initial pH of 7.10±0.1 and a final pH of 6.6. Incubation temperature 37° C. for a time of 17±1 hours. The long-term storage conditions are at −25° C. Growth is carried out in forced anaerobic conditions in a TPY broth medium at 37° C. The gram-positive strain is anaerobic and presents with rods of various forms. Non-acid consuming, non-spore forming and non-mobile. The glucose present in the medium is degraded exclusively by the metabolic pathway (shunt) of fructose-6-phosphate.

The strains referred to above were then checked for genotypic and phenotypic stability, absence of acquired and/or transmissible antibiotic resistances, resistance to gastric juices, pancreatic secretion and biliary salts, and the feasibility of production of each strain on an industrial scale.

The strains referred to above are producers of folic acid easily assimilable by the enterocytes because it is made up of a limited number of glutamylic residues (1, 2 and 3 molecules glutamic acid).

The average quantity of folic acid which the strains are capable of producing in 48 hours in the broth medium is from about 10 to over 100 ng/ml, preferably from 25 to 100 ng/ml, even more preferably from 40 to 85 ng/ml. The production of folic acid also occurs in mixed faecal cultures, thus demonstrating that it occurs at colon level in the presence of a complex microbiota, often made up of over 1,000 different species belonging to distinct genera and/or families: Lactobacillaceae, Ciostrildiaceae, Bifidobacteriaceae, Bacterioides, Enterococcaceae, Streptococcaceae, Fusobacterium, Enterobacteriaceae, Propionibacterium, Micrococcaceae, Staphilococcaceae.

### Study in an Animal Model

The Applicant has also conducted an in-vivo study with Wistar rats. These rats have been maintained on a controlled diet totally devoid of folates. The aim of the in-vivo study was to induce a deficiency and, at the same time, to check whether the administration of the strains of folic acid-producing Bifidobacteria referred to above was capable of increasing the amount of folic acid/folates in the rats.

The animals were divided into 4 groups and fed as in the table:

| | |
|---|---|
| Group 1 | Diet devoid of folates |
| Control | |
| Group 2 | Diet supplemented with a mixture of 3 strains |
| PRO | of folic acid-producing bacteria (DSM 18350, |
| | DSM 18352, DSM 18353) |
| Group 3 | Diet supplemented with fructooligosaccharides |
| PRE | (FOS) |
| Group 4 | Diet supplemented with a mixture of DSM 18350, |
| SYM | DSM 18352, DSM 18353 and |
| | fructooligosaccharides (FOS) |

All 4 groups were fed the same diet devoid of folates for the entire period of the study. The diet of Group 2 (PRO) was supplemented with a mixture of 3 folic acid-producing probiotic strains (DSM 18350, DSM 18352 and DSM 18353) at the rate of 2×10⁸ cells per strain per day; Group 3 (PRE) was fed a quantity of fructooligosaccharides (FOS) equal to 10 grams/litre of water; Group 4 (SYM) was fed both the 3 probiotic strains and the FOS in a quantity equal to what was described above for Group 2 and Group 3. The strains were administered in a ratio of 1:1:1 in a dose of 2×10⁸ cells per strain per day.

After 14 days the rats were sacrificed and the blood samples and bioptic specimens were analysed. Folic acid was determined using a biological method, much more sensitive and precise, based on the use of the test micro-organism Lactobacillus casei subsp. rhamnosus ATCC 7469. From the data present in the literature, this micro-organism is capable of growing only in the presence of folates, specifically folic acid, both in its native state and in variously reduced forms such as dihydrofolic acid (DHF), tetrahydrofolic acid (THF) and their methylated or formilated derivatives. The strain effectively uses the monoglutamate forms and, in a lesser measure, also the di- and tri-glutamate forms. A slight sensitivity was demonstrated, however, towards the polyglutamate forms.

The results are summarised in the table below:

Concentration of folic acid found after 14 days of differentiated nourishment

Concentration of folic acid found after 14 days of
differentiated nourishment

| Group | plasma ng/ml) | liver (mcg/g) | kidneys (mcg/g) |
|---|---|---|---|
| 1) Control | 2.39 | 1.09 | 0.71 |
| 2) PRO | 5.73 | 1.27 | 0.67 |
| 3) PRE | 5.85 | 1.23 | 0.71 |
| 4) SYM | 9.90 | 1.58 | 0.77 |

The values reported represent the average of those obtained for the individual animals constituting a group.

The plasmatic concentration of folic acid increased by more than 2 times for Group (2) PRO and by more than 4 times for Group (4) SYN. In this last case the importance of the prebiotic fibre is evident, as an energy source for the strains used.

In conclusion the studies executed with the animal model demonstrated that the strains are capable of colonising rats' intestines in a short time, and synthesising vitamin B₉ in quantities such as to cause it to be absorbed through the intestinal epithelium and subsequently distributed through the plasma pathway, accumulating in the liver, the organ delegated with this function in man as well.

The Applicant has also carried out an “in-vivo” study to evaluate the capacity of the three probiotic strains belonging to the two species Bifidobacterium adolescentis and Bifidobacterium pseudocatenulatum for producing folates in the human intestinal environment. The evaluation was made by means of a randomised study comprising a total of 23 healthy subjects who had followed an average varied diet.

In particular, the subjects were divided into three groups: Group A (5 subjects) was treated with the probiotic strain Bifidobacterium adolescentis DSM 18350 administered in a quantity of 5×10⁹ CFU/day; Group B (13 subjects) was treated with the probiotic strain Bifidobacterium adolescentis DSM 18352 administered in a quantity of 5×10⁹ CFU/day; and Group C (5 subjects) took the strain Bifidobacterium pseudocatenulatum DSM 18353 in a quantity of 5×10⁹ CFU/day. The capacity of the strains to colonise the intestine and produce folic acid was assessed by comparing both the number of micro-organisms belonging to the genus Bifidobacterium and the quantity of folates present in the faeces evacuated over a period of 48 hours, before and after treatment with the probiotic strains.

As a preliminary, an observational enquiry was carried out into the dietary habits of the subjects taking part in the study, with particular reference to the consumption of foods rich in folates. The base value was then determined for the concentration of folic acid/g of faeces and a calculation made of the total quantity of vitamin excreted in 48 hours. At the time of the start of the treatment the subjects were asked to maintain their dietary regime as far as possible unchanged, so as not to alter the input of exogenous folic acid.

After 30 days of administration of the probiotic strains, the new concentration of folic acid in the faeces was analysed, recalculating the total quantity of vitamin excreted in 48 hours. The difference between the two values (d30−d0) is due to the production of endogenous folic acid by the strains of Bifidobacterium which colonised the intestine.

Quantification of the vitamin was effected using the same protocol as had been adopted in the study in the animal model referred to above, where Lactobacillus rhamnosus ATCC 7469 is used as the test organism.

The table below shows the average quantities of folic acid excreted with the faeces over a 24 hour period in the three groups:

Average quantity of folic acid excreted with the faeces
over a 24 hour period

| Group | d₀ | D₃₀ | d₃₀ − d₀ | p |
|---|---|---|---|---|
| A | 98.6 ± 25.1 | 167.0 ± 28.3 | 68.4 ± 38.5 | 0.004 |
| B | 121.9 ± 31.5 | 202.2 ± 39.1 | 80.3 ± 28.7 | <0.001 |
| C | 105.2 ± 32.3 | 155.4 ± 35.9 | 50.2 ± 30.5 | 0.049 |

The results demonstrate that taking specific probiotic strains capable of producing folic acid has mediated a statistically significant increase in the concentration of this vitamin even in the faeces of all the groups treated, especially those belonging to Group B. The numerical evaluation of the Bifidobacteria present in the faeces has confirmed the potential of all the strains to colonise the intestinal environment, especially as regards B. adolescentis DSM 18352.

The studies performed demonstrate the effective capacity of the strains of bacteria of the present invention to synthesise and secrete folates including in the human intestinal environment, confirming that a preparation on the basis of these strains could represent a endogenous complementary source of vitamin B₉, which is particularly useful for the homoeostasis of the mucosal enterocytes of the colon and for ensuring the constant bioavailability of the vitamin, by contrast with what occurs when it is taken orally.

Another object of the present invention is a symbiotic supplement comprising a probiotic and a prebiotic component for use in the treatment of plasma hyperhomocysteinaemia.

The probiotic component comprises at least one strain of bacterium chosen from the group consisting of Bifidobacterium adolescentis No. DSM 16594, Bifidobacterium adolescentis No. DSM 16595, Bifidobacterium breve No. DSM 16596, Bifidobacterium pseudocatenulatum No. DSM 16597, Bifidobacterium pseudocatenulatum DSM 16598, Bifidobacterium adolescentis No. DSM 18350, Bifidobacterium adolescentis No. DSM 18352, Bifidobacterium adolescentis No. DSM 18353, or Bifidobacterium pseudocatenulatum No. DSM 21444.

The prebiotic component comprises at least one prebiotic fibre known to experts in the field, such as for example inulin, fructooligosaccharides, galactooligosaccharides, glucooligosaccharides, xylooligosaccharides, arabinogalactane, glucomannans, galactomannans and/or their combinations.

The Applicant has found that the presence of a vitamin complex of the B group, in immediately available form, capable of supplying all the vitamins and elements which preside directly or indirectly over the homocysteine cycle, enables the treatment of hyperhomocysteinaemia.

The symbiotic supplement can comprise from 0.1 to 100×10⁹ CFU/dose of bacteria and from 0.1 to 10 CFU/dose of prebiotic fibre(s). The supplement can also comprise one or more of the B group of vitamins, each present in quantities varying from 5 to 100% of the RDA.

The folic acid produced by the bacteria, together with the B group of vitamins present in the composition contribute to regulating the concentration of homocysteine, which is responsible for the increase in the risk of onset of numerous serious pathologies.

A preferred embodiment of the present invention is in the form of a dietary supplement which comprises at least one of the B group of vitamins capable of ensuring, in immediately available form, a quantity equal 50% of the RDA (Recommended Dietary Allowance) of all the vitamins and elements which preside directly or indirectly over the homocysteine cycle. The supplement also comprises at least one bacterial strain capable of synthesising folic acid at colon level, chosen from the group comprising Bifidobacterium adolescentis No. DSM 16594, Bifidobacterium adolescentis No. DSM 16595, Bifidobacterium breve No. DSM 16596, Bifidobacterium pseudocatenulatum No. DSM 16597, Bifidobacterium pseudocatenulatum No. DSM 16598, Bifidobacterium adolescentis No. DSM 18350, Bifidobacterium adolescentis No. DSM 18352, Bifidobacterium adolescentis No DSM 18353 and Bifidobacterium pseudocatenulatum No. DSM. 21444.

In a preferred embodiment the supplement contains a mixture of three strains such as Bifidobacterium adolescentis No. DSM 18350, Bifidobacterium adolescentis No. DSM 18352 and Bifidobacterium adolescentis No. DSM 18353.

In another embodiment the supplement contains Bifidobacterium adolescentis No. DSM 18352.

In addition to the B group of vitamins and the strains of bacteria, the supplement comprises at least one fibre with prebiotic bifidogenic activity. The fibre is chosen from the group comprising Inulin with a degree of polymerization (DP) of between 9 and 12 and FOS (fructooligosaccharides) with a degree of polymerization (DP) of between 2 and 4 in a quantity sufficient to ensure a prompt and continuous colonisation by the strains of bacteria present.

Both the fibres referred to above belong to the class of fructans, polysaccharides consisting of mixtures of linear polymers of fructose with various degrees of polymerization, having a unit of glucose in terminal position.

The efficacy of the above-mentioned supplement is manifested both at topical intestinal level thanks to the trophism induced by the folic acid secreted by the strains bacteria, particularly by B. adolescentis DSM 18352, on the enterocytes of the colon, and at systemic level through the lowering of the plasmatic concentration of homocysteine due to the methylation brought about by 5-methyltetrahydrofolate.

The formulation of the supplement referred to above has the following advantages.

1A) Rapid lowering of the concentration of homocysteine in the blood thanks to the B-vitamin component, present in adequate quantity (50% RDA) and in immediately bioavailable form. In particular, 5-methyltetrahydrofolate represents a type of folic acid utilisable even by individuals deficient in L-glutamyl-transferase and folate/dehydrofolate-reductase.

1B) Normalisation of the base plasmatic levels of homocysteine thanks to the synthesis of folic acid effected in the colon by the strains of bacteria present which manage to colonise the various intestinal segments thanks to the bifidogenic action of the various prebiotic fibres present.

The synthesis mechanism enables significant concentrations to be attained, in the intestinal lumen, of vitamin B₉, which, being made up of forms with a limited number of glutamyl residues, can enter the enterocytes not only by means of active transport, but also by passive diffusion through the basolateral membrane.

The constant input of endogenous folic acid, metabolised through the same pathways as exogenous folic acid, ensures a continuous supplementation of folates at systemic level, thus normalising the plasmatic concentration of homocysteine.

Consequently, there is a reduction in the risk of the onset of serious pathologies such as arteriosclerosis, stroke, cardiac infarction and neuro-degenerative diseases of great social importance, such as vascular dementia, senile depression and Alzheimer's disease.

Non-limiting example of a formulation of a supplement which is of the present invention.

| Supplement | Mg/packet | % RDA |
|---|---|---|
| Mixture of DSM | 250 | n.a. |
| 18350, DSM 18352, | | |
| DSM 18353 | | |
| Inulin | 2,000 | n.a. |
| FOS | 1,000 | n.a. |
| Vitamin B9 | 0.10 | 50% |
| Vitamin B6 | 1 | 50% |
| Vitamin B2 | 0.80 | 50% |
| Vitamin B12 | 0.001 | 50% |
| Zinc gluconate | 52 | 50% |

The presence of vitamin B₉, in quantities equal to 50% of the RDA, immediately ensures an amount effective in the reduction of the risk of onset of serious pathologies. The production of biologically significant quantities of folic acid by the probiotic strains begins only after 10-15 days, because the strains need this lapse of time to multiply and colonise the intestine. Only when their population passes a particular threshold does the production of vitamin become significant and important, ensuring a continuous input, independent of the quantity ingested with food.

The strains of bacteria of the present invention, in particular the strains DSM 18350, 18352 and 18353, have been chosen because of their capacity to produce folic acid.

| Composition 1 | Mg/packet | % RDA |
|---|---|---|
| Strain DSM 18352 | 250 | — |
| Inulin | 2,000 | — |
| FOS | 1,000 | — |
| Pyridoxine hydrochloride (Vitamin B₆) | 1 | 50 |
| Riboflavin 5-phosphate sodium | 0.8 | 50 |
| (Vitamin B₂) | | |
| Cyanocobalamine (Vitamin B₁₂) | 0.001 | 50 |
| 5-methyltetrahydrofolate (folic acid - | 0.10 | 50 |
| Vitamin B₉) | | |
| Zinc gluconate | 52 | 50 |
| Sorbitol E420 | 2,000 | — |
| Natural vanilla flavouring | 300 | — |
| Insoluble fibre | 189 | — |
| Citric acid | 20 | — |
| Sucralose E955 | 5 | — |
| TOTAL | 5,818 | |

| Composition 2 | Mg/packet | % RDA |
|---|---|---|
| Strain DSM 18352 | 250 | — |
| Inulin | 2,000 | — |
| FOS | 1,000 | — |
| Pyridoxine hydrochloride (Vitamin B₆) | 1 | 50 |
| Riboflavin 5-phosphate sodium | 0.8 | 50 |
| (Vitamin B₂) | | |
| Cyanocobalamine (Vitamin B₁₂) | 0.001 | 50 |
| 5-methyltetrahydrofolate (folic acid - | 0.10 | 50 |
| Vitamin B₉) | | |
| Zinc gluconate | 52 | 50 |
| Sorbitol E420 | 500 | — |
| Natural blackcurrant flavouring | 500 | — |
| Insoluble fibre | 139 | — |
| Citric acid | 20 | — |
| Black carrot | 100 | |
| Sucralose E955 | 10 | — |
| TOTAL | 4,573 | |

| Composition 3 | Mg/packet | % RDA |
|---|---|---|
| Mixture of DSM 18350, DSM 18352, DSM | 300 | — |
| 18353 | | |
| Inulin | 2,000 | — |
| FOS | 1,000 | — |
| Pyridoxine hydrochloride (Vitamin B₆) | 1 | 50 |
| Riboflavin 5-phosphate sodium (Vitamin | 0.8 | 50 |
| B₂) | | |
| Cyanocobalamine (Vitamin B₁₂) | 0.001 | 50 |
| 5-methyltetrahydrofolate (folic acid - | 0.10 | 50 |
| Vitamin B₉) | | |
| Zinc gluconate | 52 | 50 |
| Sorbitol E420 | 500 | — |
| Insoluble fibre | 129 | |
| Apple flavouring | 100 | — |
| Citric acid | 20 | — |
| Sucralose E955 | 5 | — |
| TOTAL | 5,008 | |

1. A composition for dietary supplements comprising at least one bacterial strain chosen from the group comprising Bifidobacterium adolescentis No. DSM 16594, Bifidobacterium adolescentis No. DSM 16595, Bifidobacterium breve No. DSM 16596, Bifidobacterium pseudocatenulatum No. DSM 16597, Bifidobacterium pseudocatenulatum DSM 16598, Bifidobacterium adolescentis No. DSM 18350, Bifidobacterium adolescentis No. DSM 18352, Bifidobacterium adolescentis No. DSM 18353 and Bifidobacterium pseudocatenulatum No. DSM 21444, in association with at least one vitamin chosen from the B group of vitamins, for the treatment of plasma hyperhomocysteinaemia.

2. The composition according to claim 1, wherein said composition comprises said Bifidobacterium adolescentis No. DSM 18352 in association with at least one bacterial strain chosen from the group comprising Bifidobacterium adolescentis No. DSM 16594, Bifidobacterium adolescentis No. DSM 16595, Bifidobacterium breve No. DSM 16596, Bifidobacterium pseudocatenulatum No. DSM 16597, Bifidobacterium pseudocatenulatum DSM 16598, Bifidobacterium adolescentis No. DSM 18350, Bifidobacterium adolescentis No. DSM 18353 and Bifidobacterium pseudocatenulatum No. DSM 21444.

3. The composition according to claim 2, wherein said composition comprises said Bifidobacterium adolescentis No. DSM 18350, Bifidobacterium adolescentis No. DSM 18352 and Bifidobacterium adolescentis No. DSM 18353.

4. The composition according to claim 3, wherein said composition consists of Bifidobacterium adolescentis No. DSM 18352.

5. The composition according to claim 1, wherein the vitamin is chosen from the group comprising vitamin B2 (riboflavin 5-phosphate sodium), vitamin B6 (pyridoxine hydrochloride), vitamin B9 (folic acid) and vitamin B12 (cyanocobalamine).

6. The composition according to claim 1, wherein said composition furthermore comprises at least one fibre with prebiotic bifidogenic activity.

7. The composition according to claim 6, wherein said fibre is chosen from the group comprising inulin and fructooligosaccharides.

8. The composition according to claim 7, wherein the inulin has a degree of polymerization of between 9 and 12 and the fructooligosaccharides have a degree of polymerization of between 2 and 4.

9. The composition according to claim 1, wherein said composition furthermore comprises at least one essential element chosen from the group comprising zinc and selenium.

10. The composition according to claim 9, wherein the zinc comes from an organic salt such as zinc gluconate or from a yeast or bacterium capable of internalising the zinc.

11. The composition according to claim 9, wherein the selenium comes from an organic salt such as zinc gluconate or from a yeast or bacterium capable of internalising the selenium.

12-22. (canceled)

23. A bacterial strain chosen from the group comprising Bifidobacterium adolescentis No. DSM 16594, Bifidobacterium adolescentis No. DSM 16595, Bifidobacterium breve No. DSM 16596, Bifidobacterium pseudocatenulatum No. DSM 16597, Bifidobacterium pseudocatenulatum DSM 16598, Bifidobacterium adolescentis No. DSM 18350, Bifidobacterium adolescentis No. DSM 18352, Bifidobacterium adolescentis No. DSM 18353 and Bifidobacterium pseudocatenulatum No. DSM 21444 for use in the treatment of plasma hyperhomocysteinaemia.

24. Use of at least one bacterial strain chosen from the group comprising Bifidobacterium adolescentis No. DSM 16594, Bifidobacterium adolescentis No. DSM 16595, Bifidobacterium breve No. DSM 16596, Bifidobacterium pseudocatenulatum No. DSM 16597, Bifidobacterium pseudocatenulatum DSM 16598, Bifidobacterium adolescentis No. DSM 18350, Bifidobacterium adolescentis No. DSM 18352, Bifidobacterium adolescentis No. DSM 18353 and Bifidobacterium pseudocatenulatum No. DSM 21444 for the preparation of a pharmaceutical composition for the treatment of plasma hyperhomocysteinaemia.

25. Use according to claim 24, wherein the pharmaceutical composition comprises said Bifidobacterium adolescentis No. DSM 18352 in association with at least one bacterial strain chosen from the group comprising Bifidobacterium adolescentis No. DSM 16594, Bifidobacterium adolescentis No. DSM 16595, Bifidobacterium breve No. DSM 16596, Bifidobacterium pseudocatenulatum No. DSM 16597, Bifidobacterium pseudocatenulatum DSM 16598, Bifidobacterium adolescentis No. DSM 18350, Bifidobacterium adolescentis No. DSM 18353 and Bifidobacterium pseudocatenulatum No. DSM 21444.

26. Use according to claim 24, wherein said at least one bacterial strain is in association with at least one vitamin chosen from the B group of vitamins.

27. Use according to claim 26, wherein said composition comprises the bacterial strain Bifidobacterium adolescentis No. DSM 18352 in association with at least one bacterial strain chosen from the group comprising Bifidobacterium adolescentis No. DSM 16594, Bifidobacterium adolescentis No. DSM 16595, Bifidobacterium breve No. DSM 16596, Bifidobacterium pseudocatenulatum No. DSM 16597, Bifidobacterium pseudocatenulatum DSM 16598, Bifidobacterium adolescentis No. DSM 18350, Bifidobacterium adolescentis No. DSM 18353 and Bifidobacterium pseudocatenulatum No. DSM 21444.

28. Use according to claim 27, wherein said composition comprises Bifidobacterium adolescentis No. DSM 18350, Bifidobacterium adolescentis No. DSM 18352 and Bifidobacterium adolescentis No. DSM 18353.

29. Use according to claim 26, wherein the vitamin is chosen from the group comprising vitamin B2 (riboflavin 5-phosphate sodium), vitamin B6 (pyridoxine hydrochloride), vitamin B9 (folic acid) and vitamin B12 (cyanocobalamine).

30. Use according to claim 26, wherein said composition furthermore comprises at least one fibre with prebiotic bifidogenic activity.

31. Use according to claim 26, wherein said composition furthermore comprises at least one essential element chosen from the group comprising zinc and selenium.
